(19) United States
(12) Patent Application Publication
(10) Pub. No.: US 2022/0000642 A1
(43) Pub. Date: Jan. 6, 2022

(54) METHOD FOR TRANSMITTING DATA

(71) Applicant: Otto Bock Healthcare Products GmbH, Wien (AT)

(72) Inventors: Sebastian Amsuess, Wien (AT); Serhii Andrushchenko, Vösendorf (AT); Fabian Osswald, Pfaffstätten (DE); Kerstin Hofmann, Wien (DE)

(73) Assignee: Otto Bock Healthcare Products GmbH, Wien (AT)

(21) Appl. No.: 17/234,682

(22) Filed: Apr. 19, 2021

(30) Foreign Application Priority Data
Apr. 20, 2020 (DE) 10 2020 110 687.4

(51) Int. Cl. A61F 2/70; A61F 2/60; H04W 4/80

(52) U.S. Cl. CPC A61F 2/70; A61F 2/60; A61B 5/4851; A61F 2002/705; H04W 4/80

(57) ABSTRACT

The invention relates to a method for transmitting data between a prosthesis system and an input device, wherein the prosthesis system has a plurality of prosthesis components which have at least one electronic and/or mechatronic component, wherein the prosthesis system is connected to the input device via at least one interface, wherein at least two electronic and/or mechatronic components, in a state when connected to the prosthesis components, are together connected to the input device, and data are transmitted or exchanged via the one input device.

The invention relates to a method by which data, in particular for configuring a prosthesis system, are transmitted between a prosthesis system and an input device, wherein the prosthesis system has a plurality of prosthesis components which have at least one electric and/or mechatronic component, wherein at least one prosthesis component is connected to an input device via an interface, and it also relates to a system for carrying out the method.

Prostheses replace missing or lost limbs. The simplest prosthesis components have a purely cosmetic function or complete a limb, for example by replacing a distal finger joint. Over the course of time, prostheses have become more complex, with multiple prosthesis components being arranged on and fastened to one another and, for example, connected to one another via joints. Complex mechanical drive devices have been developed, for example in order to move prosthetic hands or prosthetic feet. Hydraulic damping devices have been arranged on joints in order to modify the behavior of prosthesis components and prosthesis systems, so as to permit a sequence of movements that is as natural as possible. In order to assist movements, drives have been integrated in prosthesis components, such that active prostheses have been obtained. Moreover, sensors have been arranged on prosthesis components or on a prosthesis user in order to detect the current pattern of movement and to estimate the future pattern of movement and adjust the settings of dampers and/or drives. This has resulted in highly complex prosthesis systems with multiple prosthesis components arranged on one another, which have a large number of mechanical, electrical and mechatronic components.

A prosthesis system of the lower limb can have a thigh socket on which a prosthetic knee joint, a prosthetic lower leg and a prosthetic foot are fastened. Such a prosthesis system has, for example, two or more joints, which can each be provided with dampers and/or drives. Such prosthesis systems have to be individually adapted to the particular patient, which is done by an orthopedic technician. Each individual prosthesis component with the mechatronic components has to be selected for the particular user and optimally adapted. To do this, a large number of data and parameters have to be input for each prosthesis component, for example user-specific data such as weight, degree of mobility, height, and much more besides.

The object of the present invention is to make available a method and a system for transmitting data, in particular for configuring a prosthesis system, by which it is possible to achieve an improved and more rapid adaptation of the system or of different system components to the particular user.

According to the invention, this object is achieved by a method having the features of the main claim and by a system having the features of the additional independent claim. Advantageous embodiments and developments of the invention are disclosed in the subclaims, the description and the figures.

In the method for transmitting data between a prosthesis system and an input device, in particular for configuring a prosthesis system, wherein the prosthesis system has a plurality of prosthesis components which have at least one electronic and/or mechatronic component, wherein at least one prosthesis component is connected to an input device via at least one interface, provision is made that at least two electronic and/or mechatronic components, in a state when connected to the prosthesis components, are together connected to the input device, and data are transmitted or exchanged via the one input device. At present, it is necessary to configure each prosthesis component individually, for which purpose each individual prosthesis component with a mechatronic component is connected to the input device so that the necessary data are transmitted or exchanged. By way of an input mask, a large number of interrogations are carried out for each prosthesis component and, if appropriate, for each mechatronic component in order to obtain, through the data exchange or the data transmission, a configuration that is adapted to the patient. This can take a great deal of time, especially in the case of bilateral patients. According to the invention, provision is made that all of the electronic and/or mechatronic components are connected to the input device together with the prosthesis components, and a data transmission or a data exchange is carried out via the input device, resulting for example in improved adjustment convenience and enhanced user friendliness for the orthopedic technician and the patient. The time needed for the data transmission or for the data exchange, e.g. in the configuration of a prosthesis system, can be halved or reduced by up to as much as 25%. In addition, automated inputs or settings are possible for a large number of interrogations, such that there are no longer any redundant inputs for the particular components. The input device can also be a data processing device which is coupled to a prosthesis component and which is connected to sensors, for example, and on the basis of which data are transmitted to other electronic and/or mechatronic components. The data transmission or the data exchange, e.g. in the configuration, can thus be effected via an external data processing device such as a computer, tablet, mobile phone or similar, or via a data processing device integrated in a prosthesis component. A prosthetic joint can also be an input device, for example if it obtains, from sensors, information that is relevant to the pattern of movement or gait and accordingly controls the other prosthesis components. The information can include, for example, the weight of the user, the walking speed, the stride length and similar, which information can be obtained via pressure sensors, angle sensors, position sensors, moment sensors and/or IMUs. Configuration is understood in particular to mean not only the assembling of structural parts and software components, but also the setting up of the software and hardware and the coordination of the components to one another and their adaptation to one another, wherein each aspect is also to be regarded individually as configuration. The configuration of all the components can be effected via a single input device, wherein a direct data transmission or a direct data exchange, e.g. in the configuration, can take place through a connection of the components to the input device via the interfaces, or an indirect data transmission or an indirect data exchange, e.g. in the configuration, can take place via an interface to a component from which the further data transmission or the data exchange or the further configuration is effected.

In a development of the invention, provision is made that at least two prosthesis components, in particular all the prosthesis components of the prosthesis system, are fastened to each other and then provided with data and configured or a data exchange takes place. It is thereby possible to automatically detect interactions between the individual prosthesis components and their mechatronic components and take these interactions into account in the data transmission or the data exchange or the configuration. If the two prosthesis components or the respective prosthesis components are fastened to each other, this can be automatically detected in the input device by sensors, transmitters, transponders or switches. Contacts that detect the presence of the attached prosthesis component can be formed on the mechanical coupling devices, for example pyramid adapters or the like. Similarly, information can be transmitted as to which prosthesis component is arranged on the respective other prosthesis component. For each prosthesis component, different interrogations or inputs can be stored that only have to be input once in a combination of the two prosthesis components. After it has been detected that the two prosthesis components or several prosthesis components have been fastened to one another, a large number of data have to be input only a single time in order to configure the whole prosthesis system. In addition, it is possible to insert or fade out certain input fields which are needed only in the respective combination of two or more prosthesis components or are not required. Each prosthesis component can have a plurality of electronic and/or mechatronic components, which can likewise be automatically identified.

The prosthesis components can be connected to the input device via at least one wireless interface, as a result of which it is easier for the orthopedic technician and the user of the prosthesis components to perform the data transmission or the data exchange or the configuration. It is also possible in principle that the data transmission or the data exchange or the configuration is performed remotely from the patient, for example after maintenance work, or for adaptation if, on account of changes in the pattern of movement or in the abilities of the patient, another prosthesis component appears to be expedient. The new prosthesis component is then sent to the particular prosthesis user and/or to the orthopedic technician. After it has been fitted, the electric and/or mechatronic components are then optimized and configured with respect to the adjustment made and are adapted to the prosthesis components already present. The adaptation and/or the adjustment can be effected by the orthopedic technician and/or the prosthesis user.

Configuration parameters of the prosthesis system and/or instructions, in particular commands for executing movements, can be transmitted or exchanged via the input device.

Previously determined or stored user-related data, in particular for the configuration, can be transmitted via the input device to the respective prosthesis component or the prosthesis components. If user-related data are already present with the orthopedic technician or can be called up from a database, for example with the manufacturer of the prosthesis components, the physician, the physiotherapist or another institution that looks after the person using the prosthesis system, they can be stored in the input device or transmitted to the latter, such that a reduction in the input time can be achieved.

The interface to the input device can be bidirectional, such that items of information are not only transmitted from the input device to the prosthesis component or prosthesis components, but, conversely, items of information can also be transmitted to the respective input device via the prosthesis components and the electronic and/or mechatronic components located in these. These items of information can be compared with the interrogation masks stored in the input device or otherwise evaluated and/or processed. It is likewise possible that user-related data already present are automatically processed according to the respectively determined and identified prosthesis component, such that an accelerated data transmission or an accelerated data exchange or an accelerated configuration is possible. By way of the bidirectional interface, it is moreover possible that the orthopedic technician directly obtains feedback concerning the behavior of the prosthesis system as a reaction to the adjustments that have been made.

By way of the input device, the mechatronic components can be configured individually or can be configured jointly, according to the detected combination or established combination of the prosthesis components, wherein the data transmission or the data exchange or the configuration is effected via the input device. In this way, it is possible for the orthopedic technician, or for the person carrying out the data transmission or the data exchange or the configuration, to carry out an adaptation to the wishes and abilities of the particular prosthesis user.

Various parameters of the prosthesis components and of the mechatronic components can be modified via the input device, wherein the parameters or parameter ranges that can be modified are displayed on the input device according to the existing combination of prosthesis components. This facilitates the data transmission or the data exchange or the configuration, since only the parameters or parameter ranges that are actually expedient and possible are displayed. For example, different damping levels may be expedient in the stance phase damping of a prosthetic knee joint with different prosthetic feet, such that some damping ranges can or must be excluded from the outset. In this way, the danger of an incorrect adjustment or incorrect configuration is avoided or reduced.

The data transmission or the data exchange, in particular for the configuration of the electric and/or mechatronic component or components, can be effected directly via the input device, which is connected via at least one interface to the respective prosthesis component and thus to the respective electronic and/or mechatronic component. If only one interface is present or the use of only one interface is deemed advantageous, for example for saving space, or if a wireless connection to a component is desired, the data transmission or the data exchange or configuration can also take place with interposition of one or more prosthesis components or electronic and/or mechatronic components. The components are interconnected, if appropriate both mechanically and in terms of information technology. In the case of bilateral patients, all of the components are advantageously coupled via a wireless connection. The indirect data transmission or configuration then provides that the input device of the one interface is connected to one prosthesis component, and then items of information or data, instructions, commands or configuration parameters are distributed via this prosthesis component to the coupled and/or connected prosthesis components.

In the system for carrying out the method described above with a plurality of prosthesis components which have electronic and/or mechatronic components, provision is made that the prosthesis components are simultaneously connected to a single input device via at least one interface. The plurality of prosthesis components can be arranged on and fastened to one another, wherein at least two prosthesis components can be fastened to each other when a data transmission, a data exchange or a configuration takes place. The interface can be designed as a wireless interface, in particular as a bidirectional interface. In principle, it is also possible that a wired connection to one or more of the prosthesis components is present when a data transmission, a data exchange or a configuration takes place.

User-related data can be stored in the input device and can form a basis for the configuration of all of the prosthesis components and of the mechatronic components assigned to the prosthesis components. The input device can be designed as a computer, tablet or mobile phone or as a data processing device integrated in a prosthesis component.

The invention is explained in more detail below with reference to the figures, in which:

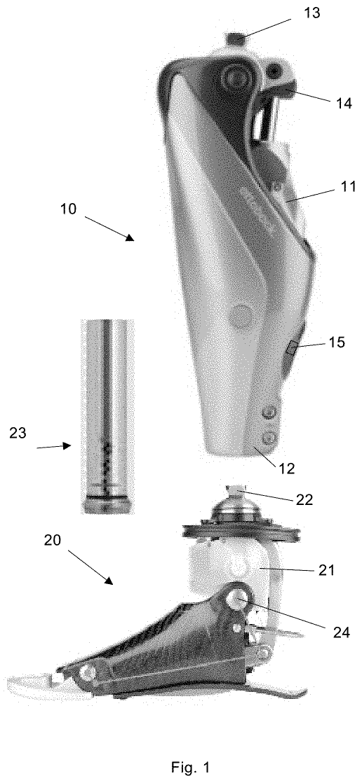

FIG. 1 shows a prosthesis system having a plurality of prosthesis components;

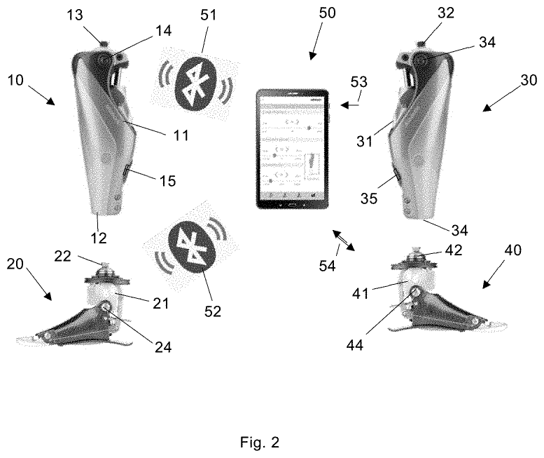

FIG. 2 shows a system composed of prosthesis system and input device; and

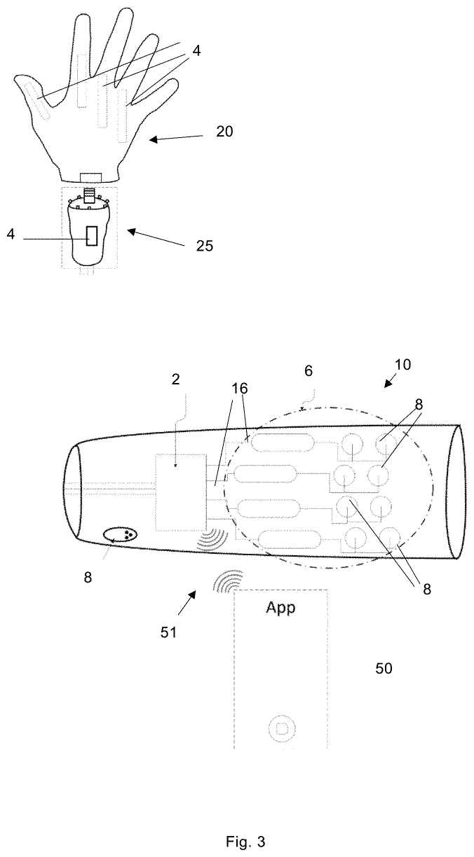

FIG. 3 shows a schematic view of an embodiment of the prosthesis system of a first prosthesis component in the form of a forearm socket.

FIG. 1 shows a side view of a prosthesis system having two prosthesis components 10, 20. A first prosthesis component 10 is designed as a prosthetic lower leg with a prosthetic knee joint. The prosthetic knee joint has an upper part, which is mounted on a lower part so as to be pivotable about a pivot axis 14. A first mechatronic component 11, which is designed as an electronically controlled hydraulic damping device, is arranged inside the lower part. Arranged inside the mechatronic component 11 are actuators which are activated on the basis of sensor data and which are arranged, for example, on the first prosthesis component 10 and are connected to a control device that is assigned to the mechatronic component 11. By way of the actuators, settings are modified or valves are closed or opened or components are actively moved. Arranged at the proximal end of the first prosthesis component 10 is a pyramid adapter 13 by which it is possible to connect the prosthetic knee joint, as part of the first prosthesis component 10, to a thigh socket (not shown). A fastening device 12 is arranged or formed at the distal end of the first prosthesis component 10 and serves to receive a pyramid adapter 22 which is arranged in the proximal end of the second prosthesis component 20, the latter in the form of a prosthetic foot. In many cases, a tube adapter 23 or lower-leg tube is needed for coupling the prosthetic knee joint 10 or the lower leg part to the prosthetic foot 20.

The prosthetic foot 20 has a foot part, an ankle part, and an ankle joint axis 24 about which the ankle part can be pivoted relative to a foot part. A further mechatronic component 21, which can likewise be controlled electronically, is arranged inside the ankle part. Dampers or drives arranged inside the prosthetic foot 20 are adjusted or activated/deactivated on the basis of sensor data. The processing of the sensor data can take place separately in the respective prosthesis components 10, 20 or jointly in a common control device, with the sensor data being processed and evaluated. The control device has at least one microprocessor which is able, during the use of the prosthesis components 10, 20, to detect, capture and process the electronic sensor data and convert these into control signals for the respective drives or actuators. Storage elements are likewise provided in the control device or in the control devices in order to store data or programs. Separate and/or common energy accumulators can be assigned to the respective prosthesis component 10, 20.

The two prosthesis components 10, 20 can be fastened mechanically to each other. This is done by inserting the proximal pyramid adapter 22 of the prosthetic foot 20 into a corresponding fastening device 12 at the distal end of the lower leg part or of the first prosthesis component 10 or of a tube adapter 23.

Markers or identifiers can be arranged in the respective prosthesis component in order to make it possible, upon mechanical connection of two prosthesis components to each other, that the respective control device is informed of or detects which combination of prosthesis components is actually present. The markers or identifiers can also be interrogated wirelessly via an input device, such that the input device knows which components are intended to be configured and for which patient. The markers can be electronic markers, e.g. RFID or transponders, optical markers in the form of QR codes or other information carriers that are automatically readable.

In the illustrative embodiment shown, a transmitting and receiving device 15 is arranged on the first prosthesis component 10, making it possible to transmit information, to receive data of a control program for example, to install changes or updates, or to supply external devices with usage data that have been captured during the use of the prosthesis. The transmitting and receiving device 15 can be designed as a wireless interface or as a wired connection, for example as a socket.

FIG. 2 shows a system according to the invention having a total of four prosthesis components 10, 20, 30, 40 with which it is possible to manage a bilateral patient. The figure shows two prosthetic feet 20, 40 and two lower-leg components 10, 30 with prosthetic knee joints and proximal connector devices 13, 32. The basic set-up of the individual components corresponds to the set-up explained with reference to FIG. 1.

For the configuration of the overall system composed of a total of four prosthesis components 10, 20, 30, 40, a single input device 50 is provided which, in the illustrative embodiment shown, is designed as a tablet. Alternative input devices 50 are possible, for example a smart phone, a computer or another “smart” device. The input device 50 functions as master, while the four prosthesis components 10, 20, 30, 40 are designed as slaves which are supplied with data or information via the input device 50. The connection between the prosthesis components 10, 20, 30, 40 and the input device 50 is via wireless interfaces 51, 52, 53, 54, which are depicted symbolically. For example, data can be transmitted via a radio connection in a license-free ISM band, with what is called a Bluetooth connection. The connection can be bidirectional. Alternatively, other wireless interfaces or also wired interfaces can be arranged in order to transmit or exchange data via data exchange devices or transmitting and receiving devices 15, 35. Such transmitting and receiving devices can also be arranged or formed on the prosthetic feet 20, 40.

Whereas in the case of previous mechatronic components it was hitherto necessary, for the data transmission, the data exchange or the configuration, to connect each individual component to the input device 50 and separately adjust it or supply it with data, provision is made, according to the invention, that all of the mechatronic components 11, 21, 31, 41 are directly or indirectly connected simultaneously to one input device 50 which is designed as a master mobile device. The orthopedic technician thus has the possibility of transmitting all the input data simultaneously to all of the prosthesis components 10, 20, 30, 40, thereby permitting rapid feedback to the user of the prosthesis system. Conversely, in the case of bidirectional communication, the orthopedic technician has the possibility of obtaining all the data from all of the prosthesis components 10, 20, 30, 40 simultaneously with one interrogation, thus obtaining rapid feedback himself via the prosthesis system. In addition, by automatic comparison of the present components in a database to which the input device has access, parameters and parameters ranges can be predefined, and multiple inputs that are no longer needed can be faded out, such that not all of the user-specific data always have to be input for all the prosthesis components 10, 20, 30, 40. Through the bidirectional interfaces, it is possible that the input device 50 detects the present and combined prosthesis components 10, 20, 30, 40 and proposes or predefines parameters and parameter ranges that are adjustable. The orthopedic technician is able to choose whether the overall system is to be adjusted or the individual prosthesis components 10, 20, 30, 40 are to be adjusted.

FIG. 3 shows a schematic view of an embodiment of the prosthesis system of a first prosthesis component 10 in the form of a forearm socket or so-called brace which serves as a model of the actual forearm socket and does not yet have the full functionality of a forearm socket. A brace of this kind serves for training purposes and does not necessarily have fastening devices for the second prosthesis component 20, which is designed in the form of a prosthetic hand. In the illustrative embodiment shown, the second prosthesis component 20 is not mechanically fastened to the brace or to the forearm socket as first prosthesis component 10 but is instead arranged on a carrier 25. The carrier 25 has connectors or an interface for control signals and can also have a drive 4 for rotatable mounting of the prosthetic hand 20 or, if appropriate, drives for movable prosthetic fingers. In the illustrative embodiment shown, three drives 4 for actuation of the prosthetic fingers and of the prosthetic thumb are arranged inside the second prosthesis component 20 in the form of a prosthetic hand and can be activated and deactivated via control signals. The first prosthesis component 10 has a first sensor arrangement 6 with a plurality of electrode pairs 8, and a control device 2 in the form of an electronic data processing device. The customary electrical and electronic components are present in the control device 2, for example storage devices, data processing devices, processors, amplifiers and energy accumulators and, if appropriate, an input means and an output means and/or a display screen. Separate energy accumulators can likewise be provided. In addition, a wireless interface 51 is set up in the control device 2, via which interface the first prosthesis component 10 can be connected to the input device 50 in order to permit data exchange either in one direction or as a bidirectional data exchange.

The drive 4 arranged in the carrier 25 serves to rotate the first prosthesis component 10 or prosthetic hand relative to a final forearm socket of the prosthesis system about the longitudinal axis of the forearm socket.

The sensor arrangement 6 arranged in the brace has four electrode pairs 8. These are designed for example as individual electrode pairs 8 that can each be fastened to the skin of a prosthesis wearer. According to a further embodiment, the electrode pairs 8 are applied to a prosthesis liner (not shown), for example adhesively affixed to or connected integrally to a liner. The electrode pairs 8 are each connected to the control device 2 via sensor lines 16. By way of the sensor lines 16, the signals detected by the electrodes 8 are conveyed to the control device 2.

Besides the illustrated electrode pairs 8 as sensors for detecting myoelectrical signals, other sensors 8 are arranged on the forearm socket and can be designed as inertial sensors, IMUs, spatial sensors, acceleration sensors, force sensors, angle sensors, temperature sensors or other sensors. Several sensors 8 that detect different measurement variables or conditions can also be arranged on the orthopedic device. Similarly, sensors 8 can be arranged in the prosthetic hand, for example position sensors which detect the position of the prosthetic fingers. The signals of the sensors 8 are evaluated in the control device E.

In the present case, the control device 2 is connected wirelessly, for example by radio, to an input device 50. For example, signals obtained from the sensor arrangement 6 can be visualized via this input device 50. The control device 2 can likewise be connected wirelessly to the prosthetic hand as the second prosthesis component. There is likewise a radio connection to the carrier 25, for example. Alternatively, there may be a wired connection between the control device 2 and the second prosthesis component 20.

There is likewise the possibility of a considerable spatial separation between the second prosthesis component 20 and the control device 2 or the first prosthesis component 10. The first prosthesis component 10 or brace can be fitted in place by the user. The second prosthesis component 20 can be coupled to the control device 2, for example over the Internet, and a configuration of the overall prosthesis system can be effected via the input device 50. The input device 50 is only connected directly to the first prosthesis component 10 or the brace; the connection to the second prosthesis component 20 is effected via the first prosthesis component 10, i.e. indirectly.

By virtue of the spatial decoupling of the prosthesis components 10, 20 from each other, it is possible for an orthopedic technician to make mechanical adjustments to the prosthetic hand 20 in his workshop, while the user makes the individual adjustments via the first prosthesis component 10.

Alternatively, provision is made that the input device 50 adopts an intermediate position between the two prosthesis components 10, 20. By virtue of the bidirectional connection of the input device 50 to the first prosthesis component 10, it is possible to transmit sensor data of the sensors 8 to the input device 50. These sensor data are processed in the input device 50 and, by way of another communication interface, are sent to the remote second prosthesis component 20, which is in the form of a benchtop prosthetic hand. The orthopedic technician or the user himself can then see, e.g. via a video connection, which sensor signals lead to which reactions or control signals and therefore to corresponding movements by the drives 4 on the second prosthesis component 20, without a complete copy having to be made of a prosthesis socket with fixed positioning of the sensors. The configuration or the data transmission or the data exchange can thus proceed in parallel with the preparation of an individual forearm socket, which advantageously saves time in the management of patients.

1. A method for transmitting data between a prosthesis system and an input device, wherein the prosthesis system has a plurality of prosthesis components which have at least one electronic and/or mechatronic component, wherein the prosthesis system is connected to the input device via at least one interface, characterized in that at least two electronic and/or mechatronic components, in a state when connected to the prosthesis components, are together connected to the input device, and data are transmitted or exchanged via the one input device.

2. The method according to claim 1, characterized in that at least two prosthesis components are fastened to each other and then configured.

3. The method according to claim 2, characterized in that the prosthesis components are connected to the input device via at least one wireless interface.

4. The method according to claim 1, characterized in that configuration parameters of the prosthesis system and/or instructions, in particular commands for executing movements, are transmitted or exchanged via the input device.

5. The method according to claim 1, characterized in that previously determined or stored user-related data, in particular for the configuration, are transmitted via the input device.

6. The method according to claim 1, characterized in that the interface is bidirectional, and items of information are transmitted via the prosthesis components to the input device.

7. The method according to claim 1, characterized in that the mechatronic components are configured individually or are configured jointly, according to the detected combination or established combination of the prosthesis components, via the input device.

8. The method according to claim 1, characterized in that various parameters are modifiable via the input device, and parameters or parameter ranges are displayed on the input device according to the existing combination of prosthesis components.

9. The method according to claim 1, characterized in that the transmission or the exchange of data, in particular for the configuration of the electric and/or mechatronic components, is effected directly via the input device or with interposition of a prosthesis component.

10. A system for carrying out the method according to claim 1, the system having a plurality of prosthesis components which have electric and/or mechatronic components and are simultaneously connected to one input device via at least one interface.

11. A system according to claim 9, characterized in that at least two prosthesis components are fastened to each other.

12. A system according to claim 9, characterized in that the interface is designed as a wireless interface, in particular as a bidirectional interface.

13. A system according to claim 9, characterized in that user-related data are stored in the input device and form a basis for the transmission and/or the exchange of data, in particular for the configuration of all of the prosthesis components.

14. A system according to claim 9, characterized in that the input device is designed as a computer, tablet or mobile phone or as a data processing device integrated in a prosthesis component.

15. A method for transmitting data between a prosthesis system and an input device, wherein the prosthesis system has a plurality of prosthesis components which have at least one electronic and/or mechatronic component, wherein the prosthesis system is connected to the input device via at least one wireless interface, characterized in that at least two electronic and/or mechatronic components, in a state when connected to the prosthesis components, are together connected to the input device, and data, including the configuration parameters of the prosthesis system, are transmitted or exchanged via the one input device.

16. The method of claim 15, wherein the data includes commands for executing movements of the prosthesis system, and wherein the data are transmitted or exchanged via the input device.

17. The method of claim 15, wherein the interface is bidirectional, and items of information are transmitted via the prosthesis components to the input device.

18. The method of claim 15, wherein the mechatronic components are configured individually or jointly, according to the detected combination or established combination of the prosthesis components via the input device.

19. The method of claim 15, wherein the transmission or exchange of data, in particular for the configuration of the electric and/or mechatronic components, is effected directly via the input device or with interposition of a prosthesis component.

20. A method for transmitting data between a prosthesis system and an input device, wherein the prosthesis system has a plurality of prosthesis components, at least two of which are fastened to each other, and which have at least one electronic and/or mechatronic component, wherein the prosthesis system is connected to the input device via at least one bidirectional wireless interface, characterized in that at least two electronic and/or mechatronic components, in a state when connected to the prosthesis components, are together connected to the input device, and data, including the configuration parameters of the prosthesis system, are transmitted or exchanged via the one input device, and wherein the input device stores user-related data.
